(19) United States
(12) Patent Application Publication
(10) Pub. No.: US 2011/0197893 A1
(43) Pub. Date: Aug. 18, 2011

(54) QUICK RELEASE HEADGEAR STRAP

(75) Inventors: HEDI ZIV, BURWOOD (AU); RIKUS SLABBERT, HAWTHORN (AU); ALEXANDER PIERI, BOX HILL NORTH (AU)

(73) Assignee: COMPUMEDICS MEDICAL INNOVATION PTY LTD, ABBOTSFORD (AU)

(21) Appl. No.: 13/020,749

(22) Filed: Feb. 3, 2011

(30) Foreign Application Priority Data
Feb. 12, 2010 (AU) 2010900563

(51) Int. Cl. A61M 16/06

(52) U.S. Cl. 128/207.13

(57) ABSTRACT

This invention provides a headgear for a mask assembly including straps for positioning on the head and means to minimise deviation from an optimal mask assembly setting when engaging or disengaging the mask assembly from operative use. Preferably, the disengaging means is a variable-length strap incorporating at least one length delimiter, which may be a hub in the strap or an alternative means which employ friction to remain in place. The strap surfaces may be modified to provide the delimiting means. The strap may incorporate a second delimiting means in moveable engagement with the strap. Preferably, one engagement means is in fixed engagement with the strap. One delimiting means may be a thickened strap portion. However, other means for delimiting the effective strap length may be used.

### RELATED APPLICATION

This application claims the priority and benefit of Australian Provisional Application No. 2010900563, filed on 12 Feb. 2010.

### FIELD OF THE INVENTION

The present invention relates to straps used in headgear for masks for the provision of gases to subjects, in particular, masks for gas delivery under pressure to the airways of a subject.

### BACKGROUND

Some treatments require the use of a mask assembly for the delivery of gas or air to a subject. For example, in the treatment of sleep apnoea, gas is often delivered at continuous positive airway pressure (CPAP) wherein gas is supplied continuously at a pressure greater than ambient, or variable positive airway pressure (VPAP) wherein gas is supplied at varying pressures, or other such methods such as BiPAP wherein gas is supplied at two pressures, or APAP wherein gas is supplied at pressures determined automatically by the delivery system to a sleeping subject through a mask to keep the patient's airways open for effective respiration. Often, gas must be delivered through a mask for sustained time periods, for example, through the whole, overnight period of sleep.

It is important for continuous therapeutic benefit from pressurised gas or air that a mask assembly be comfortable and relatively leak-proof. The mask must be comfortable so that a subject achieves the therapeutic benefit of relatively unbroken sleep periods. Masks known in the art often comprise of an interface such as a cushion or pillow, made of soft material to be most comfortable for a subject during long periods of use, but are capable of sealing adequately to minimise leaks. In mask assemblies known in the art, the masks are often fitted on a user's face or nose and held in place by a headgear comprising of fully or partially elastic straps.

Prior art mask assemblies and their harnessing straps are fitted individually to a subject to obtain an optimal setting or configuration. An optimal setting is one which minimises discomfort while delivering gas with minimal leakage.

A common user complaint with current masks is that it is complex to re-fit a mask after taking it off and ensuring the mask configuration is optimal on re-fitting. What is needed is a mask assembly incorporating a headgear with more effective retention of optimal mask configuration during repeated use.

### SUMMARY OF THE INVENTION

It is an object of the invention to provide a headgear for a mask assembly that is relatively quick and easy to release and quick and easy to re-fit in repeated use while maintaining a comfortable and relatively leak-proof configuration. The invention provides a means for engaging a mask frame that provides solutions for two problems with prior art engagement means. In one aspect, the invention provides a headgear for a mask assembly including straps for positioning on the head and means to minimise deviation from an optimal mask assembly configuration when engaging or disengaging the mask assembly from operative use. Preferably, the disengaging means is a variable-length strap. In another aspect, the invention provides a means to assist the re-engagement of the mask for operative use. Preferably the engagement means comprises of at least one length delimiter. Preferably the length delimiter comprises of a hub in the distal portion of the strap. Most preferably the re-engagement means comprises of a length delimiter adjacent to the ends of the disengaging means.

In another aspect, the invention provides a strap for a headgear of a mask assembly, the strap comprising of a first portion having a first engaging side, a second portion having a second engaging side; and a third portion for engaging a mask frame. Preferably, the strap incorporates at least one length-delimiting means. Preferably, the length-delimiting means employs friction to remain in place. The strap surfaces may be modified to provide the delimiting means. Preferably, the strap the length delimiting means is in engagement with the strap. Preferably, the strap incorporates a second delimiting means in moveable engagement with the strap. Most preferably, one delimiting means comprises of a thickened strap portion. However, other means for delimiting the effective strap length may be used. Preferably, one engagement means is in fixed engagement with the strap.

In another aspect, the invention provides a method for positioning a headgear and mask assembly for gas delivery on the head of a subject comprising of the steps of placing a headgear on the head, positioning the headgear for gas delivery; and fixing the headgear in position with a length-delimited strap. Preferably, the fixing employs friction to resist movement of the strap.

### BRIEF DESCRIPTION OF THE FIGURES

FIG. 1 shows a side perspective example of a prior art headgear and mask assembly having a strap interface.

FIG. 2 shows a perspective view of an embodiment of a strap of a headgear for a mask assembly according to the invention.

FIG. 3 shows a perspective view of another embodiment of a strap of a headgear for a mask assembly according to the invention.

### DETAILED DESCRIPTION OF THE FIGURES AND PREFERRED EMBODIMENTS

The appended figures illustrate embodiments of the invention. It will be understood that there are many other possible embodiments of the invention and that the invention is limited only by the scope of the claims appended hereto.

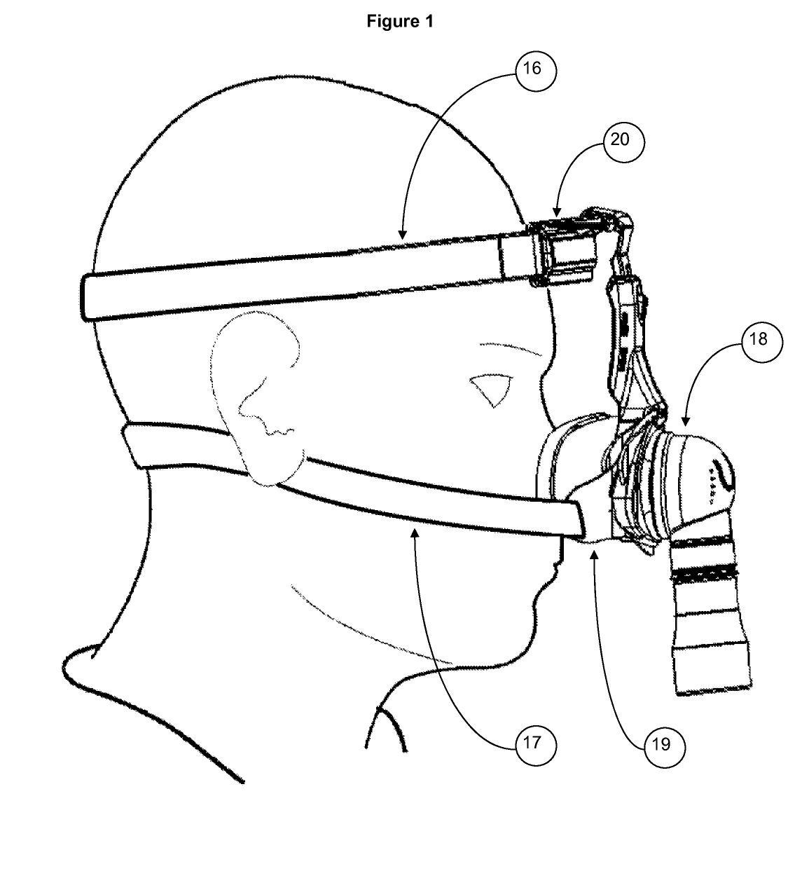

FIG. 1 shows a prior art headgear and mask assembly for positioning a nasal mask for gas delivery to a subject. The headgear comprises of straps, a mask 18 for sealing a nasal cushion against the face of a subject, the mask including a frame 19 for engaging a headgear comprising of straps 16, 17. The mask 18 is usually fitted to the user's face by at least two straps 16 & 17 in various arrangements known in the art. It will be understood that the straps 16, 17 may each encircle the head as a single strap or each strap 16, 17 comprise of a pair of straps of varying material composition and resilience, one of each pair of straps on a side of the face or head of a subject. A strap 16, 17 may engage the mask frame 19 in various methods. The present invention provides advanced solutions for headgear straps and their engagement with the mask frame or extensions thereof, such as a forehead support (such as 20 in FIG. 1), or as a mask frame (such as 19 in FIG. 1).

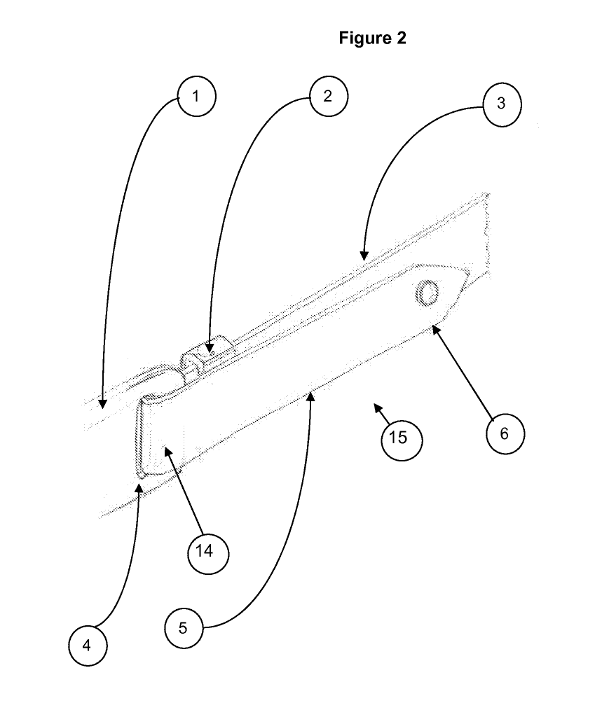

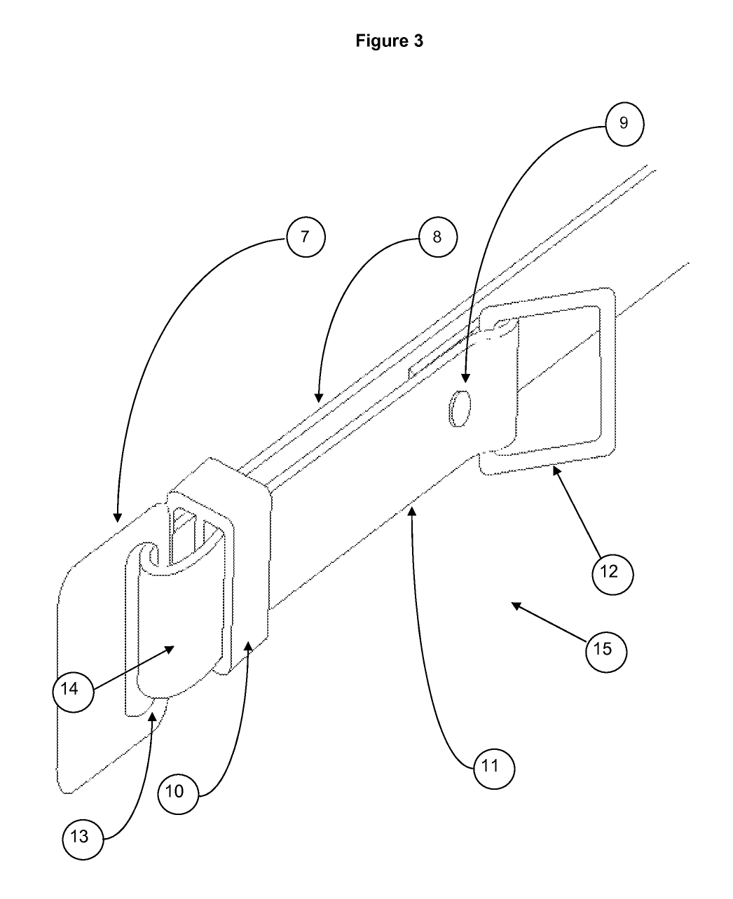

FIGS. 2 and 3 show embodiments of the invention as a strap 15 (being either or both of straps 16 or 17 of FIG. 1) comprising of a first portion 3 & 8 a second portion 5 & 11 and a third portion 14, the second portions being folded adjacent the first portions, the third portion engaged with a mask. Each of the first and second portions may include a surface for engagement with the complementary surface of the other adjacent portion. The third portion 14 engages a mask from a mask assembly (such as 19 or 20 in FIG. 1). The strap 15 may comprise of any suitable material, which may be elastic or inelastic. The second portion 5, 11 is threaded through an aperture or slot (4 FIG. 2 or 13 FIG. 3) in a mask frame or attachment (1 FIG. 2 or 7 FIG. 3) characteristically used in a mask assembly so that the third portion 14 engages the mask frame (4 FIG. 2 or 13 FIG. 3). The second portions 5, 11 of the strap 15 may be folded adjacent the first portions 3, 8. Preferably the first and second portions are in fixed engagement. The engagement surfaces of the strap portions (3 & 5 FIGS. 2 or 8 & 11 FIG. 3) may be fixedly engaged by any suitable engagement means such as Velcro™, adhesive, hook, snap and belt holes. A hook or snap, for example, may be located in one or more particular positions along the strap.

The strap 15 may incorporate at least one strap length delimiting means (2 & 6 FIGS. 2 or 10 & 12

FIG. 3). Preferably, there are two length-delimiting means. A strap delimiting means may be fixed in position or may be adjustable in relation to the strap. Preferably, an adjustable strap length-delimiting means moves in a sliding action along the axis of the strap. A delimiting means in fixed position may be a thickened portion of strap or it may be an object such as a stud, lug or the like engaged with the strap.

A strap delimiting means shown as a stud in this embodiment (6 FIGS. 2 or 9 & 12 FIG. 3), may be assembled to, penetrate, or be meshed with, the strap 15 in the second portion (5 FIG. 2 or 11 FIG. 3). The strap delimiting means is shaped or sized to prevent disengagement or unthreading, partial or otherwise, of the strap and mask frame or attachment. Embodiments of the strap delimiting means may include, without limitation, folding and sewing, bonding or gluing the edge of the strap to have a differently sized or shaped end, such as a thicker or longer end. The strap slide delimiting means (6 FIGS. 2 or 9 & 12 FIG. 3) may be thicker than the aperture or slot (4 FIG. 2 or 13 FIG. 3), thus preventing the strap from disengaging itself from the mask frame (1 FIG. 2 or 7 FIG. 3). This would most advantageously avoid the situation where the strap unthreads and a user may have to fumble to rethread the strap end into the mask frame (1 FIG. 2 or 7 FIG. 3) when trying to re-fit the mask, in the dark, during the middle of a night's sleep. Most advantageously, this invention enables a user to manipulate the strap without seeing it.

Preferably, the strap includes a second strap adjustable delimiting means (2 FIG. 2 or 10 FIG. 3), which is designed to enable adjustment along the outside surface strap 15, but has enough friction or resistance to prevent undesired movement. Another means of preventing undesired movement, while enabling a sliding feature for optimal configuration, may be a locking mechanism, such as a spring-based stopper. Preferably, the strap slide limiter, is thicker or longer than the slot (4 FIG. 2 or 13 FIG. 3), and is set during mask fitting, at a sleep clinic for instance, and then acts as an “optimal setting memory”. The optimal setting memory most advantageously operates, when the user pulls the strap while putting the mask on and fastening the straps. The strap slide limiter prevents the strap (3 FIG. 2 or 8 FIG. 3) from sliding past the optimal setting marker; the strap slide limiter (2 FIG. 2 or 10 FIG. 3) when it comes in contact with the mask frame (1 FIG. 2 or 7 FIG. 3) and slot (4 FIG. 2 or 13 FIG. 3) or a different limiter such as 10 FIG. 3.

We claim:

1. A headgear for a mask assembly comprising of:
straps for positioning on the head; and
engagement means to engage the headgear with a mask assembly, said engagement means comprising of a variable length strap.

2. The headgear of claim 1, wherein the strap incorporates at least one length delimiting means.

3. The headgear of claim 2, wherein a first delimiting means is in engagement with the strap and a second delimiting means is in adjustable engagement with the strap.

4. The headgear of claim 2 wherein a second delimiting means is in fixed engagement with the strap.

5. The headgear of claim 4 wherein one delimiting means comprises of a thickened strap portion.

6. A strap for a headgear of a mask assembly, the strap comprising of:
a first portion having a first engaging side;
a second portion having a second engaging side; and
a third portion for engaging a mask frame.

7. The strap of claim 6, further comprising at least one length delimiting means.

8. The strap of claim 7 wherein the length delimiting means is in engagement with the strap.

9. The strap of claim 7 wherein a delimiting means comprises of a thickened strap portion.

10. The strap of claim 7 wherein a delimiting means is in fixed engagement with the strap.

11. The strap of claim 7, wherein a first delimiting means is in engagement with the strap and a second delimiting means is in adjustable engagement with the strap.

12. A method for positioning a headgear and mask assembly for gas delivery on the head of a subject comprising the steps of:
(a) placing a headgear and mask assembly on the head,
(b) fitting said headgear and mask assembly for gas delivery; and
(c) adjusting the fit of said headgear with a length-delimiting means.

13. The method of claim 12 wherein the adjusting step uses frictional resistance.

14. A method for delivering pressurised gas to a subject, the method comprising of:
a) placing a headgear and mask assembly on the head,
b) fitting said headgear and mask assembly for gas delivery;
c) adjusting the fit of said headgear with a length-delimiting means; and
d) supplying pressurised gas to the mask assembly.

15. The method of claim 14 wherein the adjusting step uses frictional resistance.
